(19) United States
(12) Patent Application Publication
(10) Pub. No.: US 2024/0159760 A1
(43) Pub. Date: May 16, 2024

(54) METHOD FOR DIAGNOSING PANCREATIC CANCER

(71) Applicants: INSERM (INSTITUT NATIONAL DE LA SANTÉ ET DE LA RECHERCHE MÉDICALE), Paris (FR); INSTITUT RÉGIONAL DU CANCER DE MONTPELLIER, Montpellier (FR); UNIVERSITÉ DE MONTPELLIER, Montpellier (FR); CENTRE HOSPITALIER UNIVERSITAIRE DE MONTPELLIER, Montpellier (FR)

(72) Inventors: Andrei TURTOI, MONTPELLIER (FR); François-Régis SOUCHE, MONTPELLIER (FR); Guillaume TOSATO, MONTPELLIER (FR); Jacques COLINGE, MONTPELLIER (FR)

(21) Appl. No.: 18/550,831

(22) PCT Filed: Mar. 16, 2022
(86) PCT No.: PCT/EP2022/056858

§ 371 Date: Sep. 15, 2023

(30) Foreign Application Priority Data
Mar. 17, 2021 (EP) 21305326.7

(51) Int. Cl. G01N 33/574

(52) U.S. Cl. CPC G01N 33/57438

(57) ABSTRACT

The present invention relates to the diagnostics of pancreatic cancer. The inventors engineered a novel biomarker discovery approach, tailored for PDAC, which is all-patient inclusive, termed PanEXPEL. This approach offers access to PDAC clinical material before any treatment is applied. The method benefits from clinical biopsy, yet does not interfere with that diagnostic procedure. It can be integrated seamlessly into clinical routine, and is compatible with any type of OMICS profiling. PanEXPEL relies on the interstitial tissue fluid released from the lesion during diagnostic biopsy by endoscopic ultrasound-guided fine-needle aspiration (EUS-FNA). This is the first technique that allows both clinicians and researchers to analyze identical material in the field of proteomics biomarker research. Here, they demonstrate the potential of PanEXPEL methodology by identifying a PDAC early detection signature through proteomics and subsequent statistical learning. Thus, the present invention relates to a method for diagnosing a pancreatic cancer in a subject in need thereof comprising determining in a sample obtained from the subject the expression levels of at least one biomarker selected from the group consisting of AGR2, ANXA2, ANXA3, ANXA4, CECAM6, CYP2S1, DMBT1, KRT7, KRT8, KRT17, KRT18, KRT19, MAL2, MYH14, 0LFM4, PIGR, SERPINB5, SERPINH1, and TIMP1.

### FIELD OF THE INVENTION

The invention relates to the diagnostics of pancreatic cancer.

### BACKGROUND OF THE INVENTION

Biomarkers that are readily detectable in fluid biopsies are of the utmost importance for the diagnosis and prediction of therapeutic response in all cancers. Pancreatic ductal adenocarcinoma (PDAC) is an example of a deadly malignancy that is frequently diagnosed very late, due to the difficulty in identifying sensitive and reliable biomarkers. Indeed, it is now accepted that pancreatic cancer will typically be detected at an advanced symptomatic stage, precluding realistic chances for successful therapeutic intervention. [1, 2] In fact, PDAC has the poorest prognosis of all major cancer types.[1, 2] The only curative treatment is surgery, which is only feasible in 15-20% of cases, and even in those cases resulting in a 10-year survival rate of only 4%.[3] Recently, the value of adjuvant chemotherapy with Folfirinox (a combination of Oxaliplatin, Irinotecan, Leucovorin and 5-Fluorouracil) has been demonstrated, significantly improving the survival rate for those who undergo surgery. [4] However, this option applies only to operable patients. In all other cases (more than 80%), the tumour will generally already be metastatic or locally advanced at the time of diagnosis, resulting in a 5-year survival rate below 5% for that patient population.[4, 5] With no powerful diagnostic biomarker currently available or in prospect, pancreatic cancer will be the third leading cause of cancer-related death over the next ten years. [6] Today, only the CA19-9 marker has some clinical applications. It has been reported as discriminating patients with pancreatic cancer from both healthy controls (sensitivity 80.3%, 95% confidence interval (CI95) 77.7 to 82.6; specificity 80.2%, CI95 78.0 to 82.3) and from benign pancreatic disease (sensitivity 78.2%, CI95 72.3 to 80.4; specificity 82.8%, CI95 79.9 to 85.3).[7, 8] Whereas CA19-9 is not expressed in Lewis blood-type negative patients, the best theoretical sensitivity could not surpass 92% in European and Asian patients, and 81% in patients of African origin.[9] Despite these limitations, CA19-9 remains the best available PDAC prognostic marker, and it has utility in guiding clinicians in their adjustment of multimodal therapy and their monitoring of therapeutic response.[7] Other diagnostic tools with encouraging results are currently emerging, such as microRNAs in the blood.[10] However, there is no current means of discriminating PDAC patients from those with other pancreatic diseases (e.g., chronic pancreatitis). Overall, no current biomarker or other test procedure is deemed suitable for screening patients with no or unspecific symptoms, as is frequently the case for those with early-stage pancreatic cancer.[11]

The discovery of biomarkers, which could diagnose early-stage, treatable lesions depends on the availability of corresponding, pre-malignant material. In this regard, tissues rather than liquid biopsies are the most valuable resource. Indeed, upon their release from the tumour into the blood, biomarkers are diluted up to several billion-fold and mixed with molecular species originating from other tissues. The discovery of novel markers in these conditions has proven to be extremely challenging. In addition, the markers most useful for early diagnosis are those that originate from early, pre-cancerous lesions. Ideally, patients should have not received any treatment that might distort the native features of those lesions. Access to such material is often very limited (regardless of the pathology), and is usually made available only to the pathologist as a means of establishing a definitive diagnosis. In the case of PDAC, most patients are typically already metastatic and not operable at the time of diagnosis. Of those who are operable, a significant portion will have received heavy neo-adjuvant chemotherapy. Effectively, the proportion of operable, non-treated, early-lesion PDAC patients from whom useful biomarker research material could be obtained is less than 1%.

### SUMMARY OF THE INVENTION

The inventors engineered a novel biomarker discovery approach, tailored for PDAC, which is all-patient inclusive (i.e. applies to both operable and non-operable cases), termed PanEXPEL. This approach offers access to PDAC clinical material before any treatment is applied. The method benefits from clinical biopsy, yet does not interfere with that diagnostic procedure. It can be integrated seamlessly into clinical routine, and is compatible with any type of OMICS profiling. PanEXPEL relies on the interstitial tissue fluid released (expelled) from the lesion during diagnostic biopsy by endoscopic ultrasound-guided fine-needle aspiration (EUS-FNA). Thus, it mines the richest source of soluble, undiluted, uncontaminated biomarkers. [12-14] This is the first technique that allows both clinicians and researchers to analyze identical material in the field of proteomics biomarker research. Here, they demonstrate the potential of PanEXPEL methodology by identifying a PDAC early detection signature through proteomics and subsequent statistical learning.

Thus, the present invention relates to a method for diagnosing a pancreatic cancer in a subject in need thereof comprising determining in a sample obtained from the subject the expression levels of at least one biomarker selected from the group consisting of AGR2, ANXA2, ANXA3, ANXA4, CECAM6, CYP2S1, DMBT1, KRT7, KRT8, KRT17, KRT18, KRT19, MAL2, MYH14, OLFM4, PIGR, SERPINB5, SERPINH1, and TIMP1. Particularly, the invention is defined by its claims.

### DETAILED DESCRIPTION OF THE INVENTION

### Method for Diagnosing Pancreatic Cancer

Another aspect of the invention relates to a method for diagnosing a pancreatic cancer in a subject in need thereof comprising i) determining in a sample obtained from the subject the expression levels of at least one biomarker selected from the group consisting of AGR2, ANXA2, ANXA3, ANXA4, CECAM6, CYP2S1, DMBT1, KRT7, KRT8, KRT17, KRT18, KRT19, MAL2, MYH14, OLFM4, PIGR, SERPINB5, SERPINH1, and TIMP1 ii) comparing the expression level determined at step i) with its predetermined reference value and iii) concluding that the subject in need thereof has a pancreatic cancer when the expression level determined at step i) is higher than its predetermined reference value, or concluding that the subject in need thereof has not a pancreatic cancer when the expression level determined at step i) is lower than its predetermined reference values.

In a particular embodiment, the expression levels of 1, 2, 3, 4, 5, 6, 7, 8, 9, 10, 11, 12, 13, 14, 15, 16, 17, 18, or 19 biomarkers selected from the group consisting of AGR2, ANXA2, ANXA3, ANXA4, CECAM6, CYP2S1, DMBT1, KRT7, KRT8, KRT17, KRT18, KRT19, MAL2, MYH14, OLFM4, PIGR, SERPINB5, SERPINH1, and TIMP1 can be determined according to the method of the invention.

In a particular embodiment, the expression levels of the 19 biomarkers can be determined.

Thus, in this particular embodiment, the invention relates to a method for diagnosing a pancreatic cancer in a subject in need thereof comprising i) determining in a sample obtained from the subject the expression levels of the biomarkers selected from the group consisting of AGR2, ANXA2, ANXA3, ANXA4, CECAM6, CYP2S1, DMBT1, KRT7, KRT8, KRT17, KRT18, KRT19, MAL2, MYH14, OLFM4, PIGR, SERPINB5, SERPINH1, and TIMP1 ii) comparing the expression levels determined at step i) with their predetermined reference values and iii) concluding that the subject in need thereof has a pancreatic cancer when the expression levels determined at step i) are higher than their predetermined reference values, or concluding that the subject in need thereof has not a pancreatic cancer when the expression levels determined at step i) are lower than their predetermined reference values.

In a particular embodiment, a step of normalisation of the expression levels of the biomarkers can be realized.

In a particular embodiment, the expression levels of the biomarkers AGR2, ANXA2, ANXA3, ANXA4, CECAM6, CYP2S1, DMBT1, KRT7, KRT8, KRT17, KRT18, KRT19, MAL2, MYH14, OLFM4, PIGR, SERPINB5, SERPINH1, and TIMP1 can be log-transformed and summed to obtain a score.

In a particular embodiment, the protein expression levels of all of the 19 biomarkers can be evaluated to obtain a protein signature score (PSS) and a reference value (or cut-off or threshold) can be determined. According to the invention, here the cut-off value can be between 80 and 90 and particularly is 88.5 or 85 to augmented the positive predictive value to 100%.

In a particular embodiment, the age of the subject can be determined and can be used as a threshold.

Thus, in a particular embodiment, the invention relates to a method for diagnosing a pancreatic cancer in a subject in need thereof comprising i) determining in a sample obtained from the subject the expression levels of at least on biomarker selected from the group consisting of AGR2, ANXA2, ANXA3, ANXA4, CECAM6, CYP2S1, DMBT1, KRT7, KRT8, KRT17, KRT18, KRT19, MAL2, MYH14, OLFM4, PIGR, SERPINB5, SERPINH1, and TIMP1 ii) log-transformed and summed the different expression levels of the biomarkers to obtain a protein signature score (PSS), iii) comparing the PSS determined at step ii) with a predetermined reference values and iv) concluding that the subject in need thereof has a pancreatic cancer when the PSS is superior than 85 and when the subject is 54 years old or more or concluding that the subject in need thereof has not a pancreatic cancer when the PSS is inferior than 85.

In another embodiment, the invention relates to a method for diagnosing a pancreatic cancer in a subject in need thereof comprising i) determining in a sample obtained from the subject the expression levels of the biomarkers selected from the group consisting of AGR2, ANXA2, ANXA3, ANXA4, CECAM6, CYP2S1, DMBT1, KRT7, KRT8, KRT17, KRT18, KRT19, MAL2, MYH14, OLFM4, PIGR, SERPINB5, SERPINH1, and TIMP1 ii) log-transformed and summed the different expression levels of the biomarkers to obtain a protein signature score (PSS), iii) comparing the PSS determined at step ii) with a predetermined reference values and iv) concluding that the subject in need thereof has a pancreatic cancer when the PSS is superior than 85 and when the subject is 54 years old or more or concluding that the subject in need thereof has not a pancreatic cancer when the PSS is inferior than 85.

Another aspect of the invention relates to a method for predicting the survival time of a patient suffering from a pancreatic cancer comprising i) determining in a sample obtained from the subject the expression levels of at least one biomarker selected from the group consisting of PIGR, SPB5, ANXA3, AGR2, SERPH and ANXA4 and ii) concluding that the subject in need thereof has a bad outcome when the expression level determined at step i) is high, or concluding that the subject in need thereof has not a bad outcome when the expression level determined at step i) is low.

According to this aspect, the expression levels of the biomarkers can be compared between different patients to allow the prediction of the outcome.

In a particular embodiment, the invention relates to a method for predicting the survival time of a patient suffering from a pancreatic cancer comprising i) determining in a sample obtained from the subject the expression levels of at least one biomarker selected from the group consisting of PIGR, SPB5 and ANXA3 ii) concluding that the subject in need thereof has a bad outcome when the expression level determined at step i) is high, or concluding that the subject in need thereof has not a bad outcome when the expression levels determined at step i) is low.

In a particular embodiment, the invention relates to a method for predicting the survival time of a patient suffering from a pancreatic cancer comprising i) determining in a sample obtained from the subject the expression levels of the biomarkers selected from the group consisting of PIGR, SPB5 and ANXA3 ii) concluding that the subject in need thereof has a bad outcome when the expression levels determined at step i) are high, or concluding that the subject in need thereof has not a bad outcome when the expression levels determined at step i) are low.

In a particular embodiment, the invention relates to a method for predicting the survival time of a patient suffering from a pancreatic cancer comprising i) determining in a sample obtained from the subject the expression levels of at least one biomarker selected from the group consisting of AGR2, SERPH and ANXA4 ii) concluding that the subject in need thereof has a bad outcome when the expression levels determined at step i) is low, or concluding that the subject in need thereof has good outcome when the expression levels determined at step i) is high.

In a particular embodiment, the invention relates to a method for predicting the survival time of a patient suffering from a pancreatic cancer comprising i) determining in a sample obtained from the subject the expression levels of the biomarkers selected from the group consisting of AGR2, SERPH and ANXA4 ii) concluding that the subject in need thereof has a bad outcome when the expression levels determined at step i) are low, or concluding that the subject in need thereof has good outcome when the expression levels determined at step i) are high.

In a particular embodiment, the invention relates to a method for predicting the survival time of a patient suffering from a pancreatic cancer comprising i) determining in a sample obtained from the subject the expression levels of at least one biomarker selected from the group consisting of PIGR, SPB5, ANXA3, AGR2, SERPH and ANXA4 ii) comparing the expression levels determined at step i) with its predetermined reference value and iii) concluding that the subject in need thereof has a pancreatic cancer when the expression level determined at step i) is higher than its predetermined reference value, or concluding that the subject in need thereof has not a pancreatic cancer when the expression level determined at step i) is lower than its predetermined reference value.

In a particular embodiment, the invention relates to a method for predicting the survival time of a patient suffering from a pancreatic cancer comprising i) determining in a sample obtained from the subject the expression levels of at least one biomarker selected from the group consisting of PIGR, SPB5 and ANXA3 ii) comparing the expression level determined at step i) with its predetermined reference value and iii) concluding that the subject in need thereof has a bad outcome when the expression level determined at step i) is higher than its predetermined reference value, or concluding that the subject in need thereof has a good outcome when the expression level determined at step i) is lower than its predetermined reference value.

In another particular embodiment, the invention relates to a method for predicting the survival time of a patient suffering from a pancreatic cancer comprising i) determining in a sample obtained from the subject the expression levels of the biomarkers selected from the group consisting of PIGR, SPB5 and ANXA3 ii) comparing the expression levels determined at step i) with their predetermined reference values and iii) concluding that the subject in need thereof has a bad outcome when the expression levels determined at step i) are higher than their predetermined reference values, or concluding that the subject in need thereof has a good outcome when the expression levels determined at step i) are lower than their predetermined reference values.

In a particular embodiment, the invention relates to a method for predicting the survival time of a patient suffering from a pancreatic cancer comprising i) determining in a sample obtained from the subject the expression levels of at least one biomarker selected from the group consisting of AGR2, SERPH and ANXA4 ii) comparing the expression level determined at step i) with its predetermined reference value and iii) concluding that the subject in need thereof has a bad outcome when the expression level determined at step i) is lower than its predetermined reference value, or providing that the subject in need thereof has good outcome when the expression level determined at step i) is higher than its predetermined reference value.

In a particular embodiment, the invention relates to a method for predicting the survival time of a patient suffering from a pancreatic cancer comprising i) determining in a sample obtained from the subject the expression levels of the biomarkers selected from the group consisting of AGR2, SERPH and ANXA4 ii) comparing the expression levels determined at step i) with their predetermined reference values and iii) concluding that the subject in need thereof has a bad outcome when the expression levels determined at step i) are lower than their predetermined reference values, or concluding that the subject in need thereof has good outcome when the expression levels determined at step i) are higher than their predetermined reference values.

In a particular embodiment, the expression levels of the 6 biomarkers PIGR, SPB5, ANXA3, AGR2, SERPH and ANXA4 can be determined according to the method of prediction of the invention.

As used herein, the term “survival time” denotes the percentage of people in a study or treatment group who are still alive for a certain period of time after they were diagnosed with or started treatment for a disease, such as a pancreatic cancer (according to the invention). The survival time rate is often stated as a five-year survival rate, which is the percentage of people in a study or treatment group who are alive five years after their diagnosis or the start of treatment.

As used herein and according to the invention, the term “survival time” can regroup the term overall survival (OS).

As used herein, the term “Overall survival (OS)” denotes the time from diagnosis of a disease such as a pancreatic cancer (according to the invention) until death from any cause. The overall survival rate is often stated as a two-year survival rate, which is the percentage of people in a study or treatment group who are alive two years after their diagnosis or the start of treatment.

As used herein and according to all aspects of the invention, the term “sample” denotes, blood, peripheral-blood, serum, plasma, interstitial tissue fluid from cancer biopsy or cancer biopsy and particularly pancreatic cancer biopsy.

As used herein, a pancreatic cancer (or tumor) can be a pancreatic ductal adenocarcinoma (PDAC), a neuroendocrine tumor (NET), an acinar cell carcinoma, an adenosquamous carcinoma, a colloid carcinoma, a giant cell tumor, mucinous cystic neoplasms (MCNs), a pancreatoblastoma, a signet ring cell carcinoma, an undifferentiated tumor, an intraductal papillary mucinous neoplasm (IPMN), a pancreatic serous cystadenomas (SCNs) or a pancreatic intraepithelial neoplasia.

TABLE A
biomarkers of the inventions:

| Gene Name of the | | | |
|---|---|---|---|
| protein | Name | Protein ID (UniProt) | Gene ID (Entrez) |
| AGR2 | Anterior gradient | O95994 | 10551 |
| | protein 2 homolog | | |
| ANXA2 | Annexin A2 | P07355 | 302 |
| ANXA3 | Annexin A3 | P12429 | 306 |
| ANXA4 | Annexin A4 | P09525 | 307 |
| CECAM6 | Carcinoembryonic | P40199 | 4680 |
| | antigen-related cell | | |
| | adhesion molecule 6 | | |
| CYP2S1 | Cytochrome P450 2S | Q96SQ9 | 29785 |
| | Subfamily S Member | | |
| | 1 | | |
| DMBT1 | Deleted in malignant | Q9UGM3 | 1755 |
| | brain tumors 1 | | |
| KRT7 | Keratin, type II | P08729 | 3855 |
| | cytoskeletal 7 | | |
| KRT8 | Keratin, type II | P05787 | 3856 |
| | cytoskeletal 8 | | |
| KRT17 | Keratin, type I | Q04695 | 3872 |
| | cytoskeletal 17 | | |
| KRT18 | Keratin, type I | P05783 | 3875 |
| | cytoskeletal 18 | | |
| KRT19 | Keratin, type I | P08727 | 3880 |
| | cytoskeletal 19 | | |
| MAL2 | Myelin and | Q969L2 | 114569 |
| | Lymphocyte T cell | | |
| | differenciation | | |
| | protein 2 | | |
| MYH14 | MYosin Heavy chain | Q7Z406 | 79784 |
| | 14 | | |
| OLFM4 | Olfactomedin 4 | Q6UX06 | 10562 |
| PIGR | Polymeric | P01833 | 5284 |
| | immunoglobulin | | |
| | receptor | | |
| SERPINB5 | Maspin (mammary | P36952 | 5268 |
| | serine protease | | |
| | inhibitor) | | |
| SERPINH1 | Heat shock protein 47 | P50454 | 871 |
| TIMP1 | Tissue Inhibitor of | P01033 | 7076 |
| | Metalloproteinases 1 | | |

Measuring the expression level of the 19 biomarkers of the invention (Table A) can be done by measuring the gene expression level of the 19 biomarkers of the invention or by measuring the protein expression level of the 19 biomarkers of the invention and can be performed by a variety of techniques well known in the art.

Typically, the expression level of a gene may be determined by determining the quantity of mRNA. Methods for determining the quantity of mRNA are well known in the art. For example the nucleic acid contained in the samples (e.g., cell or tissue prepared from the subject) is first extracted according to standard methods, for example using lytic enzymes or chemical solutions or extracted by nucleic-acid-binding resins following the manufacturer's instructions. The extracted mRNA is then detected by hybridization (e.g., Northern blot analysis, in situ hybridization) and/or amplification (e.g., RT-PCR).

Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook—A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[3vinylsulfonyl)phenyl]naphthalimide-3,5 disulfonate (Lucifer Yellow VS), N-(4-anilino-1-naphthyl)maleimide, antl1ranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfor1ic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6diclllorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 nm (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6,130,101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649,138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can he detected as colored light of a specific wavelength or fluorescence. Semiconductor nanocrystals with different spectral characteristics are described in e.g., U.S. Pat. No. 6,602,671. Semiconductor nanocrystals that can he coupled to a variety of biological molecules (including dNTPs and/or nucleic acids) or substrates by techniques described in, for example, Bruchez et al., Science 281:20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can he produced that are identifiable based on their different spectral characteristics. For example, semiconductor nanocrystals can he produced that emit light of different colors based on their composition, size or size and composition. For example, quantum dots that emit light at different wavelengths based on size (565 nm, 655 nm, 705 nm, or 800 nm emission wavelengths), which are suitable as fluorescent labels in the probes disclosed herein are available from Life Technologies (Carlshad, Calif.).

Additional labels include, for example, radioisotopes (such as 3H), metal chelates such as DOTA and DPTA chelates of radioactive or paramagnetic metal ions like Gd3+, and liposomes.

Detectable labels that can be used with nucleic acid molecules also include enzymes, for example horseradish peroxidase, alkaline phosphatase, acid phosphatase, glucose oxidase, beta-galactosidase, beta-glucuronidase, or beta-lactamase.

Alternatively, an enzyme can be used in a metallographic detection scheme. For example, silver in situ hyhridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Publication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pirlkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. 1. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/01 17153.

It will be appreciated by those of skill in the art that by appropriately selecting labelled probe-specific binding agent pairs, multiplex detection schemes can he produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can he labelled with a first hapten, such as biotin, while a second probe that corresponds to a second target sequence can be labelled with a second hapten, such as DNP. Following exposure of the sample to the probes, the bound probes can he detected by contacting the sample with a first specific binding agent (in this case avidin labelled with a first fluorophore, for example, a first spectrally distinct QUANTUM DOT®, e.g., that emits at 585 nm) and a second specific binding agent (in this case an anti-DNP antibody, or antibody fragment, labelled with a second fluorophore (for example, a second spectrally distinct QUANTUM DOT®, e.g., that emits at 705 nm). Additional probes/binding agent pairs can he added to the multiplex detection scheme using other spectrally distinct fluorophores. Numerous variations of direct, and indirect (one step, two step or more) can he envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In a particular embodiment, the methods of the invention comprise the steps of providing total RNAs extracted from cumulus cells and subjecting the RNAs to amplification and hybridization to specific probes, more particularly by means of a quantitative or semi-quantitative RT-PCR (or q RT-PCR).

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

Expression level of a gene may be expressed as absolute expression level or normalized expression level. Typically, expression levels are normalized by correcting the absolute expression level of a gene by comparing its expression to the expression of a gene that is not a relevant for determining the cancer stage of the subject, e.g., a housekeeping gene that is constitutively expressed. Suitable genes for normalization include housekeeping genes such as the actin gene ACTB, ribosomal 18S gene, GUSB, PGK1, TFRC, GAPDH, GUSB, TBP and ABL1. This normalization allows the comparison of the expression level in one sample, e.g., a subject sample, to another sample, or between samples from different sources.

According to the invention, the level of the 19 biomarkers proteins of the invention may also be measured and can be performed by a variety of techniques well known in the art. For measuring the expression level of said biomarkers proteins, techniques like ELISA (see below) allowing to measure the level of the soluble proteins are particularly suitable.

In the present application, the “level of protein” or the “protein level expression” or the “protein concentration” means the quantity or concentration of said protein. In another embodiment, the “level of protein” means the level of the biomarkers proteins fragments of the invention. In still another embodiment, the “level of protein” means the quantitative measurement of the biomarkers proteins expression of the invention relative to a negative control.

Typically protein concentration may be measured for example by capillary electrophoresis-mass spectroscopy technique (CE-MS) or ELISA performed on the sample.

Such methods comprise contacting a sample with a binding partner capable of selectively interacting with proteins present in the sample. The binding partner is generally an antibody that may be polyclonal or monoclonal, preferably monoclonal.

The presence of the protein or fragments of the proteins can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, capillary electrophoresis-mass spectroscopy technique (CE-MS), HPLC-MS, MALDI-MS etc. The reactions generally include revealing labels such as fluorescent, chemioluminescent, radioactive, enzymatic labels or dye molecules, or other methods for detecting the formation of a complex between the antigen and the antibody or antibodies reacted therewith.

The aforementioned assays generally involve separation of unbound protein in a liquid phase from a solid phase support to which antigen-antibody complexes are bound. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with a set of antibodies against the proteins to be tested. A sample containing or suspected of containing the marker protein is then added to the coated wells. After a period of incubation sufficient to allow the formation of antibody-antigen complexes, the plate(s) can be washed to remove unbound moieties and a detectably labeled secondary binding molecule is added. The secondary binding molecule is allowed to react with any captured sample marker protein, the plate is washed and the presence of the secondary binding molecule is detected using methods well known in the art.

Methods of the invention may comprise a step consisting of comparing the proteins and fragments concentration in circulating cells with a control value. As used herein, “concentration of protein” refers to an amount or a concentration of a transcription product, for instance the biomarkers of the invention. Typically, a level of a protein can be expressed as nanograms per microgram of tissue or nanograms per milliliter of a culture medium, for example. Alternatively, relative units can be employed to describe a concentration. In a particular embodiment, “concentration of proteins” may refer to fragments of the biomarkers of the invention. Thus, in a particular embodiment, fragment of the biomarkers of the invention protein may also be measured.

As used herein, the term “Protein Signature Score” (PSS) consist of the sum of the normalized, log 10-transformed expression values of proteins in a sample. In some embodiments, the PSS is obtained with the protein expression level of at least one biomarker selected in the group consisting of AGR2, ANXA2, ANXA3, ANXA4, CECAM6, CYP2S1, DMBT1, KRT7, KRT8, KRT17, KRT18, KRT19, MAL2, MYH14, OLFM4, PIGR, SERPINB5, SERPINH1, and TIMP1. In some embodiments, the PSS is obtained with the protein expression level of at least AGR2. In some embodiments, the PSS is obtained with the protein expression level of all the biomarkers selected in the group consisting of AGR2, ANXA2, ANXA3, ANXA4, CECAM6, CYP2S1, DMBT1, KRT7, KRT8, KRT17, KRT18, KRT19, MAL2, MYH14, OLFM4, PIGR, SERPINB5, SERPINH1, and TIMP1.

In some embodiments, the formula for determining PSS is:

$$PSS = {\sum }_{i = 1}^{{N}_{p}} {log}_{10} ( 1 + {\hat{X}}_{ij} )$$

Wherein Nₚ is the number of proteins included in the PSS as biomarker, each protein expression level Xᵢⱼ (protein i in the sample j) is normalized as \hat{X}ᵢⱼ after

$${\hat{X}}_{ij} = \frac{{X}_{ij}}{{\tilde{X}}_{j}} \times \frac{{\sum }_{j = 1}^{n} {\tilde{X}}_{j}}{n}$$

Wherein \tilde{X}_{J} is the median protein expression level in sample (j) and n is the number of samples.

In some embodiments, the formula for determining PSS is:

$$PSS = {\sum }_{i = 1}^{{N}_{p}} {log}_{10} ( 1 + {\hat{X}}_{ij} )$$

Wherein Nₚ is the number of proteins included in the PSS as biomarker, each protein expression level Xᵢⱼ (protein i in the sample j) is normalized as \hat{X}ᵢⱼ after

$${\hat{X}}_{ij} = \frac{{X}_{ij}}{{\tilde{X}}_{j}} \times \frac{{\sum }_{j = 1}^{n} {\tilde{X}}_{j}}{n}$$

Wherein \tilde{X}_{J} is the median of total protein expression level in sample (j) and n is the number of samples.

As used herein, the term “total protein expression level” denotes the expression level of all proteins in a sample, all protein combined. In some embodiments, the protein expression level is the concentration of said protein in a sample.

Predetermined reference values used for comparison of the expression levels may comprise “cut-off” or “threshold” values that may be determined as described herein. Each reference (“cut-off”) value for the biomarkers levels may be predetermined by carrying out a method comprising the steps of:
a) providing a collection of samples from subjects suffering of a pancreatic cancer;
b) determining the level of the biomarkers of the invention for each sample contained in the collection provided at step a);
c) ranking the tumor tissue samples according to said level or combine all the expression levels of the biomarkers of the invention to obtain a score;
d) classifying said samples in pairs of subsets of increasing, respectively decreasing, number of members ranked according to their expression level,
e) providing, for each sample provided at step a), information relating to the actual clinical outcome for the corresponding pancreatic cancer subject;
f) for each pair of subsets of samples, obtaining a Kaplan Meier percentage of survival curve;
g) for each pair of subsets of samples calculating the statistical significance (p value, sensitivity, specificity, AUC or Youden index) between both subsets
h) selecting as reference value for the level, the value of level for which the p value is the smallest.

For the method of prediction according to the invention, the p-value can be calculated as well as others parameters (AIC, BIC, LLR, etc.).

For example the expression level of the biomarkers of the invention has been assessed for 100 pancreatic cancer samples of 100 subjects. The 100 samples are ranked according to their expression level. Sample 1 has the best expression level and sample 100 has the worst expression level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding pancreatic cancer subject, Kaplan Meier curves are prepared for each of the 99 groups of two subsets. Also for each of the 99 groups, the p value between both subsets was calculated.

The reference value is selected such as the discrimination based on the criterion of the minimum p value is the strongest. In other terms, the expression level corresponding to the boundary between both subsets for which the p value is minimum is considered as the reference value. It should be noted that the reference value is not necessarily the median value of expression levels.

In routine work, the reference value (cut-off value) may be used in the present method to discriminate pancreatic cancer samples and therefore the corresponding subjects.

Kaplan—Meier curves of percentage of survival as a function of time are commonly used to measure the fraction of subjects living for a certain amount of time after treatment and are well known by the man skilled in the art.

The man skilled in the art also understands that the same technique of assessment of the expression level of a protein should of course be used for obtaining the reference value and thereafter for assessment of the expression level of a protein of a subject subjected to the method of the invention.

A further object of the invention relates to kits for performing the methods of the invention, wherein said kits comprise means for measuring the expression level of the biomarkers of the invention.

The kits may include probes, primers macroarrays or microarrays as above described. For example, the kit may comprise a set of probes as above defined, usually made of DNA, and that may be pre-labelled. Alternatively, probes may be unlabelled and the ingredients for labelling may be included in the kit in separate containers. The kit may further comprise hybridization reagents or other suitably packaged reagents and materials needed for the particular hybridization protocol, including solid-phase matrices, if applicable, and standards. Alternatively the kit of the invention may comprise amplification primers that may be pre-labelled or may contain an affinity purification or attachment moiety. The kit may further comprise amplification reagents and also other suitably packaged reagents and materials needed for the particular amplification protocol.

In another aspect and as shown by the inventors, the interstitial fluid obtained after collecting a biopsy using a fine needle (as used herein “the interstitial tissue fluid from cancer biopsy”) can be cytopreservated in a liquid called Preservcyte® (see Material & Method) and can be used to make a diagnostic of a cancer or to determine the outcome of patient suffering from a cancer. Indeed, this liquid that which is normally discarded in clinical practice can contains the interstitial tissue liquid and thus different molecules that can be used for diagnosis or biomarker discovery. Thus, said interstitial tissue fluid from cancer or the Preservcyte® can be used to detect new biomarkers (protein, circulating fragments of DNA or RNA for example) which will be used to make a diagnostic of a cancer or to determine the outcome of a patient suffering of a cancer. Technics like proteomics or transcriptomics can thus be done on the Preservcyte®.

As used herein, “Preservcyte®” is a methanol containing buffered solution.

According to this aspect, any fixative containing buffered solution that could be used to preserve the biopsy can be used according to the invention, such as: methanol, ethanol, isopropanol, or others alcohols buffered solutions or hydrophilic organic solvents buffered solutions.

As used herein a “buffered solution” denotes a solution of methanol (or any alcohol) and water which is buffered to achieve physiological salt and pH conditions like for example the commercially buffered solution Preservcyte®.

Thus, in another aspect, the invention also relates to the interstitial tissue fluid from cancer biopsy for use in the diagnostic or prognostic of a cancer.

In a particular embodiment, the invention also relates to the interstitial tissue fluid from cancer biopsy for use in the detection of new biomarkers useful to make a diagnostic of a cancer or to determine the outcome of a patient suffering from a cancer.

In a particular embodiment, the invention also relates to the Preservcyte for use in the diagnostic or prognostic of a cancer.

In another particular embodiment, the invention also relates to the Preservcyte for use in the detection of new biomarkers useful to make a diagnostic of a cancer or to determine the outcome of a patient suffering from a cancer.

Particularly, the cancer is a pancreatic cancer.

The invention also relates to a method for treating a pancreatic cancer in a subject diagnosed as having a pancreatic cancer as described above comprising the administration to said subject an anti-pancreatic cancerous agent.

The invention also relates to a method for treating a pancreatic cancer in a subject with a bad outcome (or prognostic) as described above comprising the administration to said subject an anti-pancreatic cancerous agent.

Anti-pancreatic cancer agents may be Melphalan, Vincristine (Oncovin), Cyclophosphamide (Cytoxan), Etoposide (VP-16), Doxorubicin (Adriamycin), Liposomal doxorubicin (Doxil) and Bendamustine (Treanda).

Others anti-cancer agents may be for example cytarabine, anthracyclines, fludarabine, gemcitabine, capecitabine, methotrexate, taxol, taxotere, mercaptopurine, thioguanine, hydroxyurea, cyclophosphamide, ifosfamide, nitrosoureas, platinum complexes such as cisplatin, carboplatin and oxaliplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epimbicm, 5-fluorouracil, taxanes such as docetaxel and paclitaxel, leucovorin, levamisole, irinotecan, estramustine, etoposide, nitrogen mustards, BCNU, nitrosoureas such as carmustme and lomustine, vinca alkaloids such as vinblastine, vincristine and vinorelbine, imatimb mesylate, hexamethyhnelamine, topotecan, kinase inhibitors, phosphatase inhibitors, ATPase inhibitors, tyrphostins, protease inhibitors, inhibitors herbimycm A, genistein, erbstatin, and lavendustin A. In one embodiment, additional anticancer agents may be selected from, but are not limited to, one or a combination of the following class of agents: alkylating agents, plant alkaloids, DNA topoisomerase inhibitors, anti-folates, pyrimidine analogs, purine analogs, DNA antimetabolites, taxanes, podophyllotoxin, hormonal therapies, retinoids, photosensitizers or photodynamic therapies, angiogenesis inhibitors, antimitotic agents, isoprenylation inhibitors, cell cycle inhibitors, actinomycins, bleomycins, MDR inhibitors and Ca2+ ATPase inhibitors.

Additional anti-cancer agents may be selected from, but are not limited to, cytokines, chemokines, growth factors, growth inhibitory factors, hormones, soluble receptors, decoy receptors, monoclonal or polyclonal antibodies, mono-specific, bi-specific or multi-specific antibodies, monobodies, polybodies.

Additional anti-cancer agent may be selected from, but are not limited to, growth or hematopoietic factors such as erythropoietin and thrombopoietin, and growth factor mimetics thereof.

In the present methods for treating cancer the further therapeutic active agent can be an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoemanolamine, alizapride, azasetron, benzquinamide, bietanautine, bromopride, buclizine, clebopride, cyclizine, dunenhydrinate, diphenidol, dolasetron, meclizme, methallatal, metopimazine, nabilone, oxypemdyl, pipamazine, scopolamine, sulpiride, tetrahydrocannabinols, thiefhylperazine, thioproperazine and tropisetron. In a preferred embodiment, the antiemetic agent is granisetron or ondansetron.

In another embodiment, the further therapeutic active agent can be an hematopoietic colony stimulating factor. Suitable hematopoietic colony stimulating factors include, but are not limited to, filgrastim, sargramostim, molgramostim and epoietin alpha.

In still another embodiment, the other therapeutic active agent can be an opioid or non-opioid analgesic agent. Suitable opioid analgesic agents include, but are not limited to, morphine, heroin, hydromorphone, hydrocodone, oxymorphone, oxycodone, metopon, apomorphine, nomioiphine, etoipbine, buprenorphine, mepeddine, lopermide, anileddine, ethoheptazine, piminidine, betaprodine, diphenoxylate, fentanil, sufentanil, alfentanil, remifentanil, levorphanol, dextromethorphan, phenazodne, pemazocine, cyclazocine, methadone, isomethadone and propoxyphene. Suitable non-opioid analgesic agents include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

In yet another embodiment, the further therapeutic active agent can be an anxiolytic agent. Suitable anxiolytic agents include, but are not limited to, buspirone, and benzodiazepines such as diazepam, lorazepam, oxazapam, chlorazepate, clonazepam, chlordiazepoxide and alprazolam.

In yet another embodiment, the further therapeutic active agent can be a checkpoint blockade cancer immunotherapy agent.

Typically, the checkpoint blockade cancer immunotherapy agent is an agent which blocks an immunosuppressive receptor expressed by activated T lymphocytes, such as cytotoxic T lymphocyte-associated protein 4 (CTLA4) and programmed cell death 1 (PDCD1, best known as PD-1), or by NK cells, like various members of the killer cell immunoglobulin-like receptor (KIR) family, or an agent which blocks the principal ligands of these receptors, such as PD-1 ligand CD274 (best known as PD-L1 or B7-H1).

Typically, the checkpoint blockade cancer immunotherapy agent is an antibody.

In some embodiments, the checkpoint blockade cancer immunotherapy agent is an antibody selected from the group consisting of anti-CTLA4 antibodies, anti-PD1 antibodies, anti-PDL1 antibodies, anti-PDL2 antibodies, anti-TIM-3 antibodies, anti-LAG3 antibodies, anti-IDO1 antibodies, anti-TIGIT antibodies, anti-B7H3 antibodies, anti-B7H4 antibodies, anti-BTLA antibodies, and anti-B7H6 antibodies.

In another embodiment, the invention relates to a method for treating a pancreatic cancer in a subject diagnosed as having a pancreatic cancer as described above comprising the use to said subject of radiotherapy, heavy ion treatment, brachy-radiotherapy or radio-immunotherapy.

The invention will be further illustrated by the following figures and examples. However, these examples and figures should not be interpreted in any way as limiting the scope of the present invention.

### FIGURES

FIG. 1: (A) Protein Signature Score (PSS) sorted from minimum to maximum value observed in the cohort correlated with the PDAC versus non PDAC patient status. Threshold (bold line) and 95% confidence intervals (hatched area) are shown. These estimations are based on (B) optimal PSS thresholds according to Youden index at each step of the bootstrap procedure.

FIG. 2: Decision tree resulting from recursive partitioning. Sample size with (circle) condition applied and (white) proportion of non-PDAC patients and (black) PDAC patients.

TABLE 1
Pre-EUS FNA clinical and biological characteristics of 58 patients with
suspected pancreatic lesions. Mean ± sd, two-samples one-sided nonparametric
Wilcoxon rank sum test without continuity correction. Frequency (proportion),
two samples one-sided Fisher's exact test. CRP = C-reactive protein;
PDAC = Pancreas ductal adenocarcinoma; IPMN = Intraductal papillary
mucinous neoplasm; BT = Benign tumor; CP = Chronic pancreatitis;
NET = Neuroendocrine tumor; Ratio N/L = Ratio Neutrophils/Lymphocytes;
Ratio T/L = Ratio Thrombocytes/Lymphocyte; PSS = PDAC signature score.

| ALL | PDAC | Non PDAC | Test |
|---|---|---|---|
| n = 58 | n = 43 | n = 15 | p |
| | Stage | Condition | |

| | | | |
|---|---|---|---|
| I | 3 (7%) | IPMN | 4 (27%) |
| II | 14 (33%) | BT | 3 (20%) |
| III | 10 (23%) | CP | 3 (20%) |
| IV | 16 (37%) | NET | 3 (20%) |

| Age [years] | 66.6 ± 11.7 | 69.1 ± 9.5 | 59.4 ± 14.5 | 0.021 |
|---|---|---|---|---|
| Age ≥70 [years] | 24 (41%) | 19 (44%) | 5 (33%) | 0.337 |
| Gender (men) | 39 (67%) | 29 (67%) | 10 (67%) | 0.597 |
| Head of the pancreas | 44 (76%) | 31 (72%) | 13 (87%) | 0.221 |
| Surgery | 16 (28%) | 15 (35%) | 1 (6.7%) | 0.032 |
| | n = 30 | n = 22 | n = 8 | |
| Leucocytes [G · L⁻¹] | 7.5 ± 2.8 | 7.6 ± 3.0 | 7.2 ± 2.6 | 0.426 |
| Leucocytosis | 6 (20%) | 4 (18%) | 2 (25%) | 0.52 |
| (>10 G · L⁻¹) | | | | |
| | n = 29 | n = 21 | n = 8 | |
| Thrombocytes [G · L⁻¹] | 260.3 ± 208.9 | 210.7 ± 97.8 | 390.5 ± 347.2 | 0.051 |
| Thrombopenia | 6 (21%) | 5 (24%) | 1 (12.5%) | 0.457 |
| (<150 G · L⁻¹) | | | | |
| | n = 26 | n = 20 | n = 6 | |
| Bilirubin [μmol · L⁻¹] | 80.8 ± 129.3 | 102.8 ± 140.7 | 7.4 ± 1.7 | 0.003 |
| Bilirubin >17 | 13 (50%) | 13 (65%) | 0 | 0.015 |
| μmol · L⁻¹ | | | | |
| | n = 24 | n = 19 | n = 5 | |
| CRP [mg · L⁻¹] | 17.1 ± 23.3 | 17.4 ± 24.3 | 16.3 ± 21.5 | 0.322 |
| CRP >5 mg · L⁻¹ | 10 (42%) | 8 (42%) | 2 (40%) | 0.668 |
| | n = 24 | n = 18 | n = 6 | |
| Albumin [g · L⁻¹] | 40.2 ± 6.2 | 38.9 ± 6.4 | 44.1 ± 3.5 | 0.019 |
| Albumin <38 g · L⁻¹ | 7 (29%) | 6 (33%) | 1 (17%) | 0.414 |
| | n = 23 | n = 18 | n = 5 | |
| Lymphocytes [G · L⁻¹] | 1.65 ± 0.68 | 1.56 ± 0.73 | 1.95 ± 0.37 | 0.109 |
| Lymphopenia (<1 | 5 (22%) | 5 (28%) | 0 | 0.255 |
| G · L⁻¹) | | | | |
| Neutrophils [G · L⁻¹] | 4.65 ± 1.91 | 4.81 ± 1.96 | 4.09 ± 1.76 | 0.263 |
| Neutrophilia (>7.5 | 3 (13%) | 3 (17%) | 0 | 0.461 |
| G · L⁻¹) | | | | |
| | n = 22 | n = 17 | n = 5 | |
| Ratio N/L | 3.5 ± 2.5 | 4.0 ± 2.7 | 2.1 ± 0.7 | 0.036 |
| Ratio T/L | 170.0 ± 135.0 | 154.3 ± 83.8 | 223.2 ± 251.0 | 0.319 |
| | | n = 11 | | |
| CA 19.9 [UI · mL⁻¹] | | 773.6 ± 1163.8 | | |
| | | n = 10 | | |
| ACE [UI] | | 19.76 ± 33.67 | | |

### EXAMPLE

Material & Methods

Sample Collection and Study Design

The study was approved by the Ethics Committees of the Montpellier University Hospital (IRB/CHU, Montpellier, France) and the ICM cancer clinic (CORT/ICM, Montpellier, France). During sample analysis, informed consent was specifically obtained for the research protocol from all patients. This study was registered and given a trial number (NCT03791073). All patients reported with suspect pancreatic lesions in the period from May to September 2017 at the Montpellier University Hospital and the ICM were recruited in the present study (58 patients). The patients underwent a primary endoscopic ultrasound (EUS) using the GF-UCT180 Curvilinear Array Ultrasound Gastrovideoscope (Olympus®, USA) and trans-gastric/transduodenal fine needle aspiration (FNA) using mostly a 22G needle (range 19 to 25G). Once the fine needle aspiration±biopsy was performed, the contents of the needle was rinsed in a preservation solution called ThinPrep® Cytolyt Solution (RD-01612; Hologic Inc., Marlborough, United States). The sample was sent to the pathology department. It was then centrifuged (150 g for 10 min) in order to keep the pellet that was transferred into PreservCyt® (Hologic Inc., Marlborough, United States), vortexed and inserted in the ThinPrep 5000 processor (Hologic Inc., Marlborough, United States). A glass slide was loaded into the Processor. A gentle dispersion step mixed the cell sample by currents in the fluid that are strong enough to separate debris and disperse mucus, but gentle enough to have no adverse effect on cell appearance. The cells were then captured on a filter that is specifically designed to collect cells. A thin layer of cells was then transferred to a glass slide in a 20 mm-diameter circle. This slide was analysed by the pathologist to reach the final diagnosis of the pancreatic mass.

The remaining cell-free PreservCyt® liquid was transferred to the laboratory on ice and stored at +4° C. for 2 weeks. The liquid was then processed for proteomics analysis as per description below. The pre-EUS FNA clinical and biological characteristics of individual patients involved in the current study were collected retrospectively. In order to reach a definitive diagnosis, a minimum follow-up of 2 years was ensured for all patients.

Pre-Processing of PreservCyt® Samples

PreservCyt® liquid was centrifuged at 20.000 g for 10 min at +4° C. The recovered protein pellets were solubilized in lysis buffer containing 50 mM Tris and 1% SDS, pH 8. The solubilisation of the pellet was enhanced using vortexing for 1 min. Subsequently, the samples were subjected to 5 min centrifugation at 10.000 g at +4° C. Cleared protein extracts were collected for further analysis. Protein quantification was conducted using BCA quantification Kit (Cat.: #23225, Pierce, Thermo Scientific, Rockford, IL, USA). Protein extracts were stored at −80° C. pending proteomic analysis.

Proteomic Analysis

A volume corresponding to 100 μg of protein lysate was subjected to reduction of disulfide bridges in 1,4-dithiothreitol (10 mM) (Cat.: #D0632-10G, Sigma-Aldrich, St. Louis, MO, USA), for 30 minutes at 60° C. Free cysteine thiols were then alkylated using 2-chloroacetamide (22 mM) (Cat.: #30208220, Sigma-Aldrich), for 30 minutes at RT and in darkness. Proteins were purified by adding one volume of 10% trichloroacetic acid (TCA) followed by incubation of the sample for 30 minutes on ice. The samples were then centrifuged at full speed for 10 minutes, while the resulting protein pellet was washed two times in 1 mL of cold acetone (−20° C.) and centrifuged at full speed for 5 minutes at 4° C. Pellets were cautiously air-dried for 2 minutes at RT then re-suspended in 50 μL 0.1% RapiGest SF solution in 50 mM NH4HCO3 (Cat.: #186001861, Waters, Waters Corporation, 34 Maple Street, Milford, MA 01757). After adjusting pH to 7.5, 1 μg of trypsin (Cat.: #29341524, Promega, Madison, WI, USA) was added to the samples. The protein mixture was incubated at 37° C. for overnight digestion. The following day, 0.5 μg of fresh trypsin was added to each sample and the digestion was extended at 37° C. for an additional 4 hours. Next, the samples were acidified with trifluoroacetic acid (TFA), at final concentration of 0.5%, and then incubated for 1 hour at 37° C. Samples were further centrifuged at 13 000 g for 10 minutes and the supernatants were collected. Following this, 10% of each sample was transferred to a new tube where all the samples were mixed in a library. The remaining samples were dried using Speed Vac. The library sample was further subjected to peptide fractionation using a High pH Reversed-Phase Peptide Fractionation Kit, according to the manufacturer's instructions (Thermo Fisher; cat. no.: 84868). 8 individual peptide fractions were derived from the library sample; these were then dried using Speed Vac. All samples were dissolved in water containing 2% acetonitrile and 0.5% TFA. Peptides were then desalted using OMIX C18 tips (Cat.: A57003100K, Agilent Technologies, USA) according to the manufacturer's protocol. To lock the retention times between individual runs, samples and library fractions were spiked at 25 fmol/μL with PepCalMix (Cat.: #5045759, Sciex, Singapore).

The peptide containing samples were analyzed using the 1D-nano-HPLC system (Sciex, Framingham, MA, USA), which was connected on-line with a Q-TOF mass spectrometer 5600 (Sciex). One microgram of sample was injected on the C18 analytical column (Acclaim® 75 μm×150 mm, p/n: 162224; Dionex, California, USA) with a gradient of 0-40% phase B (90% acetonitrile, 9.9% water and 0.1% formic acid) for 100 minutes at the flow rate of 0.3 μl/min. Two acquisition modes were used, data dependent acquisition (DDA) for the measurement of the library, and SWATH (Sequential Window Acquisition of All Theoretical Mass Spectra) acquisition for the samples. In the DDA mode, MS data were acquired over a range from 400 to 1600 m/z. One full MS scan and 30 MS/MS scans of the most intensive peptides found in this mass range (bearing +2 or +3 charges) were conducted per cycle. The acquired data for each fraction of the library sample were merged and used for MS/MS database search with Protein Pilot software (Sciex). For the SWATH acquisition, the DDA method was adapted by using the automated method generator embedded in the Analyst software (Sciex). Protein identification and quantification was conducted using Peak View software, based on the SWATH library.

Statistical Software

All the statistical analyses were realized with R v3.6.0.

Clinical Data Analysis

For continuous covariates, we applied a two-sample nonparametric Wilcoxon rank sum test, one-sided without continuity correction. The sample sizes ranged from 58 to 22 patients, depending on the number of missing values when recovering clinical parameters from patient records. These data were also transformed into binary covariates by following the thresholds recommended by medical authorities and learned societies for their routine interpretation. We apply two-sample, one-sided Fisher's exact test on the clinical binary covariate set.

Proteomic Data and Signature

The raw proteomic data were studied. Normalization of the data was achieved applying factors, which made the median protein expression values identical in every sample. Normalized protein expression values were subsequently log 10-transformed. The implementation of SAM was available as an R package samr.[15] We first optimized SAM parameters (number of permutations, false discovery rate (FDR)) to distinguish PDAC versus non-PDAC samples. We used the Leave One-Out Cross-Validation (LOOCV) resampling method for this purpose (500 iterations), because of its speed.[26] This resulted in a number of permutations of each SAM instance set to 200, and SAM FDR to 2.5%. We then applied SAM 500 times using subsampling with the 0.632+ rule (without replacement, preserving the proportion of PDAC versus non-PDAC patients).[27, 28] To constitute a PDAC signature from these 500 protein selections, we retained those that were selected in at least 80% of the 500 subsamples. A signature score was computed by summing the log 10-transformed normalized MS signals of the selected proteins.

The R package pROC enabled us to determine the signature score AUC, and its Youden index, which is considered as the threshold that optimizes the separation between two sample classes (PDAC and non-PDAC here).[29] CIs were determined by bootstrapping, using 1,000 resamplings with replacement. We also estimated sensitivity and specificity parameters by performing the same empirical bootstrap.

Integration of the Signature Score with Clinical Data

To combine clinical data with the PDAC signature, we employed logistic regression using the R package glm and Wald's test. Covariates with more than 20% missing values were discarded. In the case of multivariate logistic models, P-values were corrected using the Bonferroni method. In order to study the link between continuous covariates, we first tested the absence of correlation between them (using the Pearson coefficient) and then used a regression tree analysis to obtain a rule that instantiated their relationship with PDAC status (R package rpart).

Survival Analysis

We employed Cox regression using the R survival and rms packages. Clinical covariates with more than 20% missing values were discarded. Covariate Log-linearity was verified, as were the Martingale and Schoenfeld residuals. Continuous covariates that did not fulfill the Log-linearity requirement were transformed into binary covariates. We applied Wald's test and corrected P-values using the Bonferroni method in the case of multivariate models.

Results

Patient Cohort and Sample Collection

Our cohort consisted of 58 patients, all of whom underwent EUS-FNA. Pathologists could not diagnose 7 of those cases, either because only normal tissue was recovered (1 case), or because samples were devoid of cells (6 cases). Those 7 patients underwent additional EUS-FNAs until a diagnosis could be made. It is important to note that only the initial EUS-FNAs were used for PanEXPEL in every case. Most patients were PDAC (n=43, 74%, Table 1). Four out of the 7 patients who could not be initially diagnosed were finally diagnosed with PDAC, while the remaining 3 patients were revealed as having non-cancerous lesions. Of all PDAC patients, 16 (37%) were diagnosed as late stage (T4), and only 3 (7%) as early, intraductal papillary mucinous neoplasia (IPMN) type (data not shown). In the PDAC cases, the mean CA19-9 and ACE were 773.6±1163.8 (n=11) and 19.76±33.67 (n=10), respectively. Fifteen patients had benign/borderline pancreatic lesions (normal pancreatic tissue, n=2); neuroendocrine tumor (NET, n=3); undegenerated IPMN (n=4); chronic pancreatitis (CP, n=3); stromal tumor (n=3). Most of the cases analyzed, including those suffering from benign pancreatic diseases, were diagnosed with a mass in the head of the pancreas (Table 1). Based on the available clinical data, PDAC diagnosis was significantly associated with an elderly population (P=0.021), an elevated rate of serum bilirubin (P=0.003, n=26), and an elevated neutrophils/lymphocytes ratio (P=0.036, n=22). The level of serum albumin was lower in PDAC compared to non-PDAC patients (P=0.019, n=24). A similar observation was made for the number of thrombocytes (P=0.051, n=29).

Protein Contents of Residual PreservCyt® Samples and Proteomic Analysis

It is now standard practice worldwide for patients with suspected pancreatic cancer to undergo a EUS-FNA biopsy. Once the needle biopsy is collected, the content of the needle is rinsed in a ThinPrep® solution called Cytolyt®. This solution primarily serves to lyse the red blood cells found in the sample. Following mild centrifugation, cells and tissue fragments are collected in a pellet that is then transferred in the fixation solution called PreservCyt®. The pathologist filters the PreservCyt® liquid to isolate the cells/tissue fragments needed for diagnosis using the ThinPrep 5000 System®. The remnant PreservCyt® filtrate is normally discarded, but we realised that its composition (consisting of 30% methanol) would in fact be ideal for preserving and denaturing proteins. Indeed, the initial storage of the PreservCyt® at +4° C. for 14 days (the maximal legal period for diagnosis in France) led to the denaturing of all soluble proteins washed out of the biopsy. Evidence of this was shown in the fact that centrifugation of PreservCyt® resulted in the formation of a protein pellet. Solubilisation of these proteins in SDS containing buffer, followed by BCA quantification, showed that the median protein quantity recovered from a single PreservCyt® was 1953 μg in PDAC samples, and 2190 μg in non-PDAC samples (this difference was not significant; P=0.824, Wilcoxon rank sum test). Following protein extraction, the samples underwent further preparation according to standard shotgun proteomics procedures. These included reduction and alkylation reactions, as well as protein precipitation to remove contaminants and to prepare the sample for tryptic digestion. Preliminary analysis using standard, data-dependent MS acquisitions, suggested that the samples were quite complex (data not shown). This led us to opt for a SWATH (Sequential Window Acquisition of all THeoretical mass spectra) approach on a q-TOF (quadrupole time of flight) instrument. The resulting analysis identified and quantified 2,538 proteins (data not shown). While other proteomic strategies could certainly have provided larger numbers of identified proteins than our samples, SWATH enabled us to follow those 2,538 proteins across all samples systematically (no missing values). This provided a convenient basis for biomarker discovery.

Identification of a PDAC Versus a Non-PDAC Protein Signature.

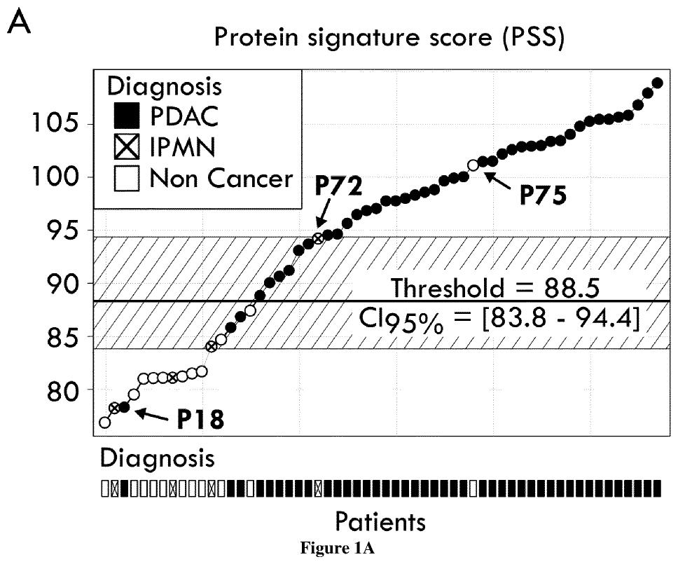

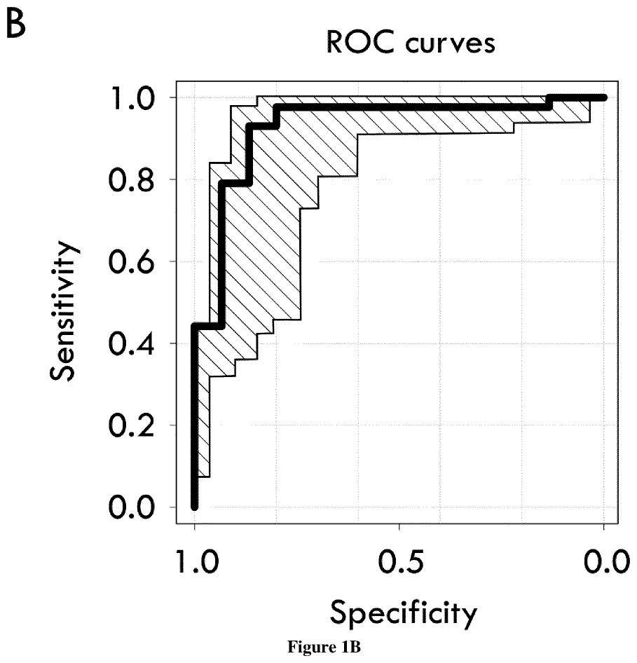

A robust protein signature discriminating PDAC from non-PDAC samples was searched using supervised statistical learning with the Significance Analysis of Microarrays (SAM) algorithm,[15] which also applies to label-free, log-transformed quantitative proteomic data. We first optimized SAM selection parameters, then employed this optimal SAM following a subsampling approach (see Experimental section below for details). Subsampling was repeated 500 times, and we kept those proteins selected by SAM in at least 80% of the subsamples. Such a procedure is commonly used to ensure robustness. It resulted in a 19-protein PDAC signature consisted of AGR2, ANXA2, ANXA3, ANXA4, CECAM6, CYP2S1, DMBT1, KRT7, KRT8, KRT17, KRT18, KRT19, MAL2, MYH14, OLFM4, PIGR, SERPINB5, SERPINH1, and TIMP1. Next, we developed a protein signature score (PSS), which consisted of the sum of the normalized, log 10-transformed expression values of the 19 proteins in each sample. PSS was significantly higher (two-samples one-sided non-parametric Wilcoxon rank sum test p<10-4) in PDAC patients (98.8±6.60 [UI]) compared to non-PDAC patients (83.7±6.4 [UI]) (FIG. 1A). PSS was independent of any clinical parameter (non-parametric Wilcoxon and Kruskal-Wallis rank sum tests P>0.70 for all the binary covariates; Pearson correlation test P>0.15 for all the continuous covariates). The Youden index (a common tool to find score thresholds optimizing both sensitivity and selectivity) yielded a PSS threshold of 88.5 (CI95 83.8-94.4 [UI]) (FIG. 1A). Using the Youden index to determine the optimal threshold at each resampling step, bootstrap estimations resulted in 0.853 specificity (CI95 0.667-0.933), and 0.917 sensitivity (CI95 0.791-0.977), with an estimated area under the curve (AUC) of 0.927 (CI95 0.827-0.995) (FIG. 1B). The PSS led to two false positives (FPs) and one false negative (FN). This performance was better than EUS-FNA, where four patients were FNs (data not shown). Three of these patients were properly diagnosed using the PanEXPEL-derived PSS.

Integrating PDAC Signature with Clinical Parameters Improves the Positive Predictive Value.

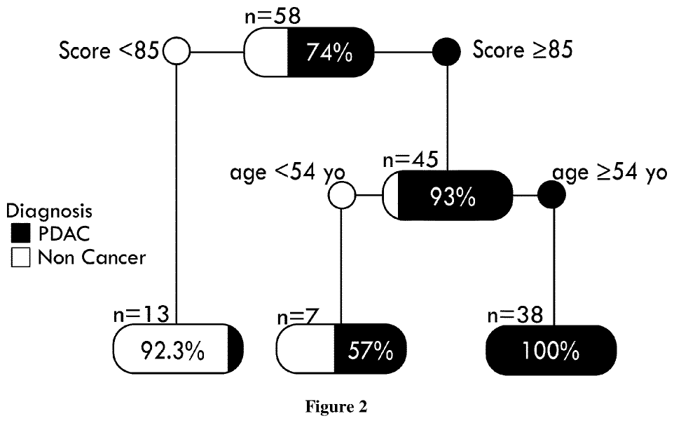

To further improve the diagnostic power of PSS, we explored using it in combination with other clinical data. Logistic regression of individual variables showed that only the age and PSS were associated with the positive diagnosis of PDAC (data not shown, age: OR 1.16, P=0.024, and PSS: OR 1.39, P=0.0004). As expected, age and PDAC were not correlated. Partition tree regression (or recursive partitioning) resulted in a decision tree (FIG. 2). While the negative and positive predictive values of PSS alone were 92.3%, and 93%, respectively, the decision tree reduced the PSS threshold to 85 (instead of 88.5—both values according to the Youden index), and augmented the positive predictive value to 100%. The optimal (Youden index) age threshold in the tree classifier was at 54.

PDAC Signature Proteins have Both Clinical and Biological Relevance.

Correlation analysis of the 19 proteins included in the PDAC signature revealed three main groups (data not shown). Groups 1 and 3 consisted of strongly correlated proteins associated with cell growth (development and differentiation) and with immune system-related processes (neutrophil degranulation and cytokine/interleukin signalling) (data not shown). Group 2 was smaller and less correlated. It contained proteins related to both immunity (PIGR) and cell growth/development (K1C17), as well as two proteins with unique functions (SERPH, ECM organization, and SPB5, negative regulation of endopeptidase activity). Although the PDAC signature was constructed with the sole goal of detecting early PDAC cases, we also tested whether these proteins have a predictive value for survival. We naturally restricted the analysis to the 43 PDAC patients, as the inclusion of non-PDAC patients would have induced a trivial association with survival. Our test employed Cox models, and found that two clinical parameters were associated with survival: age and gender (data not shown). Indeed, the hazard ratio of poor survival significantly increased with age (over 70 years old, HR: 2.62 [1.13-6.07]; P=0.011), and male gender (HR: 3.72 [1.24-11.1]; P=0.027). We therefore established a first—basal—bivariate Cox model using clinical parameters only. Then, we searched for individual proteins which, when added as a third covariate to the basal model, would significantly increase the predictive value (data not shown). We did not observe a common survival link amongst the proteins composing the signature, nor amongst the correlated groups. Moreover, most of these proteins did not significantly improve the basal bivariate Cox model. A final multivariate Cox model was built which included age, gender, and all the PDAC signature proteins that significantly improved the basal model (data not shown). Six proteins showed an impact on prognosis, three on survival loss (PIGR, SPB5, ANXA3), and three on survival gain (AGR2, SERPH, ANXA4).

### REFERENCES

Throughout this application, various references describe the state of the art to which this invention pertains. The disclosures of these references are hereby incorporated by reference into the present disclosure.
1 R. L. Siegel, K. D. Miller, and A. Jemal, Cancer statistics, 2018. CA Cancer J Clin. 2018, 68, 29313949
2 D. P. Ryan, T. S. Hong, and N. Bardeesy, Pancreatic adenocarcinoma. N Engl J Med. 2014, 371, 25427123
3 A. Paniccia, P. Hosokawa, W. Henderson, R. D. Schulick, B. H. Edil, M. D. McCarter, and C. Gajdos, Characteristics of 10-Year Survivors of Pancreatic Ductal Adenocarcinoma. JAMA Surg. 2015, 150, 26062046
4 T. Conroy, P. Hammel, M. Hebbar, M. Ben Abdelghani, A. C. Wei, J. L. Raoul, L. Chone, E. Francois, P. Artru, J. J. Biagi, T. Lecomte, E. Assenat, R. Faroux, M. Ychou, J. Volet, A. Sauvanet, G. Breysacher, F. Di Fiore, C. Cripps, P. Kavan, P. Texereau, K. Bouhier-Leporrier, F. Khemissa-Akouz, J. L. Legoux, B. Juzyna, S. Gourgou, C. J. O'Callaghan, C. Jouffroy-Zeller, P. Rat, D. Malka, F. Castan, J. B. Bachet, G. Canadian Cancer Trials, and G. I. P. G. the Unicancer, FOLFIRINOX or Gemcitabine as Adjuvant Therapy for Pancreatic Cancer. N Engl J Med. 2018, 379, 30575490
5 J. P. Neoptolemos, J. Kleeff, P. Michl, E. Costello, W. Greenhalf, and D. H. Palmer, Therapeutic developments in pancreatic cancer: current and future perspectives. Nat Rev Gastroenterol Hepatol. 2018, 15, 29717230
6 L. Rahib, B. D. Smith, R. Aizenberg, A. B. Rosenzweig, J. M. Fleshman, and L. M. Matrisian, Projecting cancer incidence and deaths to 2030: the unexpected burden of thyroid, liver, and pancreas cancers in the United States. Cancer Res. 2014, 74, 24840647
7 J. C. Gui, W. L. Yan, and X. D. Liu, CA19-9 and CA242 as tumor markers for the diagnosis of pancreatic cancer: a meta-analysis. Clin Exp Med. 2014, 14, 23456571
8 K. E. Poruk, D. Z. Gay, K. Brown, J. D. Mulvihill, K. M. Boucher, C. L. Scaife, M. A. Firpo, and S. J. Mulvihill, The clinical utility of CA 19-9 in pancreatic adenocarcinoma: diagnostic and prognostic updates. Curr Mol Med. 2013, 13, 23331006
9 E. M. Vestergaard, H. O. Hein, H. Meyer, N. Grunnet, J. Jorgensen, H. Wolf, and T. F. Orntoft, Reference values and biological variation for tumor marker CA 19-9 in serum for different Lewis and secretor genotypes and evaluation of secretor and Lewis genotyping in a Caucasian population. Clin Chem. 1999, 45, 9895338
10 A. S. Bauer, A. Keller, E. Costello, W. Greenhalf, M. Bier, A. Borries, M. Beier, J. Neoptolemos, M. Buchler, J. Werner, N. Giese, and J. D. Hoheisel, Diagnosis of pancreatic ductal adenocarcinoma and chronic pancreatitis by measurement of microRNA abundance in blood and tissue. PLoS One. 2012, 7, 22511932
11 M. Porta, X. Fabregat, N. Malats, L. Guarner, A. Carrato, A. de Miguel, L. Ruiz, M. Jariod, S. Costafreda, S. Coll, J. Alguacil, J. M. Corominas, R. Sola, A. Salas, and F. X. Real, Exocrine pancreatic cancer: symptoms at presentation and their relation to tumour site and stage. Clin Transl Oncol. 2005, 7, 15960930
12 N. Fogh-Andersen, B. M. Altura, B. T. Altura, and O. Siggaard-Andersen, Composition of interstitial fluid. Clin Chem. 1995, 41, 7586528
13 P. Gromov, I. Gromova, C. J. Olsen, V. Timmermans-Wielenga, M. L. Talman, R. R. Serizawa, and J. M. Moreira, Tumor interstitial fluid—a treasure trove of cancer biomarkers. Biochim Biophys Acta. 2013, 1834, 23416532
14 M. P. Pavlou, and E. P. Diamandis, The cancer cell secretome: a good source for discovering biomarkers? J Proteomics. 2010, 73, 20394844
15 V. G. Tusher, R. Tibshirani, and G. Chu, Significance analysis of microarrays applied to the ionizing radiation response. Proc Natl Acad Sci USA. 2001, 98, 11309499
16 S. Yachida, S. Jones, I. Bozic, T. Antal, R. Leary, B. Fu, M. Kamiyama, R. H. Hruban, J. R. Eshleman, M. A. Nowak, V. E. Velculescu, K. W. Kinzler, B. Vogelstein, and C. A. Iacobuzio-Donahue, Distant metastasis occurs late during the genetic evolution of pancreatic cancer. Nature. 2010, 467, 20981102
17 N. L. Anderson, and N. G. Anderson, The human plasma proteome: history, character, and diagnostic prospects. Mol Cell Proteomics. 2002, 1, 12488461
18 M. Gronborg, T. Z. Kristiansen, A. Iwahori, R. Chang, R. Reddy, N. Sato, H. Molina, O. N. Jensen, R. H. Hruban, M. G. Goggins, A. Maitra, and A. Pandey, Biomarker discovery from pancreatic cancer secretome using a differential proteomic approach. Mol Cell Proteomics. 2006, 5, 16215274
19 T. B. Schaaij-Visser, M. de Wit, S. W. Lam, and C. R. Jimenez, The cancer secretome, current status and opportunities in the lung, breast and colorectal cancer context. Biochim Biophys Acta. 2013, 1834, 23376433
20 J. Korf, K. D. Huinink, and G. A. Posthuma-Trumpie, Ultraslow microdialysis and microfiltration for in-line, on-line and off-line monitoring. Trends Biotechnol. 2010, 28, 20079945
21 T. Farrah, E. W. Deutsch, G. S. Omenn, D. S. Campbell, Z. Sun, J. A. Bletz, P. Mallick, J. E. Katz, J. Malmstrom, R. Ossola, J. D. Watts, B. Lin, H. Zhang, R. L. Moritz, and R. Aebersold, A high-confidence human plasma proteome reference set with estimated concentrations in PeptideAtlas. Mol Cell Proteomics. 2011, 10, 21632744
22 L. Dumartin, W. Alrawashdeh, S. M. Trabulo, T. P. Radon, K. Steiger, R. M. Feakins, M. P. di Magliano, C. Heeschen, I. Esposito, N. R. Lemoine, and T. Crnogorac-Jurcevic, ER stress protein AGR2 precedes and is involved in the regulation of pancreatic cancer initiation. Oncogene. 2017, 36, 27941872
23 K. Sogawa, S. Takano, F. Iida, M. Satoh, S. Tsuchida, Y. Kawashima, H. Yoshitomi, A. Sanda, Y. Kodera, H. Takizawa, R. Mikata, M. Ohtsuka, H. Shimizu, M. Miyazaki, O. Yokosuka, and F. Nomura, Identification of a novel serum biomarker for pancreatic cancer, C4b-binding protein alpha-chain (C4BPA) by quantitative proteomic analysis using tandem mass tags. Br J Cancer. 2016, 115, 27657339
24 P. Arumugam, S. Bhattacharya, J. Chin-Aleong, M. Capasso, and H. M. Kocher, Expression of polymeric immunoglobulin receptor and stromal activity in pancreatic ductal adenocarcinoma. Pancreatology. 2017, 17, 28173980
25 C. Tian, K. R. Clauser, D. Ohlund, S. Rickelt, Y. Huang, M. Gupta, D. R. Mani, S. A. Carr, D. A. Tuveson, and R. O. Hynes, Proteomic analyses of ECM during pancreatic ductal adenocarcinoma progression reveal different contributions by tumor and stromal cells. Proc Natl Acad Sci USA. 2019, 116, 31484774
26 B. Bischl, O. Mersmann, H. Trautmann, and C. Weihs, Resampling methods for meta-model validation with recommendations for evolutionary computation. Evol Comput. 2012, 20, 22339368
27 R. De Bin, S. Janitza, W. Sauerbrei, and A. L. Boulesteix, Subsampling versus bootstrapping in resampling-based model selection for multivariable regression. Biometrics. 2016, 72, 26288150
28 B. Efron, and T. R., Improvements on Cross-Validation: The 632+ Bootstrap Method. JASA. 1997, 92, 10.1080/01621459.1997.10474007
29 M. D. Ruopp, N. J. Perkins, B. W. Whitcomb, and E. F. Schisterman, Youden Index and optimal cut-point estimated from observations affected by a lower limit of detection. Biom J. 2008, 50, 18435502

1. A method for diagnosing a pancreatic cancer in a subject in need thereof and treating the subject, comprising i) determining in a sample obtained from the subject an expression levels of at least one biomarker selected from the group consisting of AGR2, ANXA2, ANXA3, ANXA4, CECAM6, CYP2S1, DMBT1, KRT7, KRT8, KRT17, KRT18, KRT19, MAL2, MYH14, OLFM4, PIGR, SERPINB5, SERPINH1, and TIMP1 and ii) treating a subject identified as having an expression level of the at least one biomarker that is higher than a predetermined reference value by administering to the subject at least one treatment for pancreatic cancer.

2. The method according to claim 1 wherein expression level(s) of AGR2, ANXA2, ANXA3, ANXA4, CECAM6, CYP2S1, DMBT1, KRT7, KRT8, KRT17, KRT18, KRT19, MAL2, MYH14, OLFM4, PIGR, SERPINB5, SERPINH1, and TIMP1 are determined.

3. The method according to claim 1 comprising i) determining in a sample obtained from the subject expression levels of the biomarkers AGR2, ANXA2, ANXA3, ANXA4, CECAM6, CYP2S1, DMBT1, KRT7, KRT8, KRT17, KRT18, KRT19, MAL2, MYH14, OLFM4, PIGR, SERPINB5, SERPINH1, and TIMP1 ii) comparing each expression levels determined at step i) with a predetermined reference values and iii) concluding that the subject has pancreatic cancer when the expression levels determined at step i) are higher than the predetermined reference values, or concluding that the subject in need thereof does not have a pancreatic cancer when the expression levels determined at step i) are lower than their predetermined reference values.

4. The method according to claim 1 wherein expression levels of the biomarkers AGR2, ANXA2, ANXA3, ANXA4, CECAM6, CYP2S1, DMBT1, KRT7, KRT8, KRT17, KRT18, KRT19, MAL2, MYH14, OLFM4, PIGR, SERPINB5, SERPINH1, and TIMP1 are log-transformed and summed to obtain a score.

5. The method according to claim 1 wherein the age of the subject is determined and is used as a threshold.

6. The method according to claim 5, comprising i) determining in a sample obtained from the subject expression levels of the biomarkers selected from the group consisting of AGR2, ANXA2, ANXA3, ANXA4, CECAM6, CYP2S1, DMBT1, KRT7, KRT8, KRT17, KRT18, KRT19, MAL2, MYH14, OLFM4, PIGR, SERPINB5, SERPINH1, and TIMP1 ii) log-transforming and summing the expression level determined at step i) to obtain a protein signature score (PSS), iii) comparing the PSS determined at step ii) with a predetermined reference values and iv) concluding that the subject has a pancreatic cancer when the PSS is higher than 85 or concluding that the subject does not have a pancreatic cancer when the PSS is less than 85, wherein the subject is at least 54 years old.

7. A method for predicting the survival time of a patient suffering from a pancreatic cancer and treating the patient comprising i) determining in a sample obtained from the subject an expression levels of at least one biomarker selected from the group consisting of PIGR, SPB5, ANXA3, AGR2, SERPH and ANXA4 and ii) treating a subject identified as having an expression level of the at least one biomarker that is higher than a corresponding reference value, by administering to the subject at least one treatment for pancreatic cancer.

8. The method according to claim 7 comprising i) determining in a sample obtained from the subject an expression levels of at least one biomarker selected from the group consisting of PIGR, SPB5 and ANXA3 and ii) treating the subject when the expression level is higher than a corresponding reference value, by administering to the subject at least one treatment for pancreatic cancer.

9. The method according to claim 7, further comprising determining an expression levels of at least one biomarker selected from the group consisting of AGR2, SERPH and ANXA4 ii) concluding that the subject has a poor prognosis when the expression level(s) determined at step i) is/are low, or concluding that the subject has a good prognosis when the expression level(s) determined at step i) is/are high.

10. (canceled)

11. (canceled)

12. (canceled)

13. (canceled)

14. (canceled)

15. (canceled)
